(19) United States
(12) Patent Application Publication
(10) Pub. No.: US 2025/0228893 A1
(43) Pub. Date: Jul. 17, 2025

(54) BROAD SPECTRUM ANTISEPTIC NANO EMULSION BASED ON A COMBINATION OF IONIC LIQUID AND COPPER NANOCLUSTERS

(71) Applicants: Indereshwar Singh Paul, Solan (IN); Qudrat Paul, Solan (IN); Narinder Singh, Punjab (IN)

(72) Inventors: Indereshwar Singh Paul, Solan (IN); Qudrat Paul, Solan (IN); Narinder Singh, Punjab (IN); Sanjeev Saini, Punjab (IN)

(21) Appl. No.: 18/703,178

(22) PCT Filed: Oct. 22, 2022
(86) PCT No.: PCT/IN2022/050941

§ 371 Date: Apr. 19, 2024

(30) Foreign Application Priority Data
Oct. 22, 2021 (IN) 202111048265

(51) Int. Cl. A61K 33/44; A61K 9/08; A61K 33/34; A61K 47/10; A61K 47/20; A61P 31/02

(52) U.S. Cl. CPC A61K 33/44; A61K 9/08; A61K 33/34; A61K 47/10; A61K 47/20; A61P 31/02

(57) ABSTRACT

The disclosure relates to a novel antiseptic formulation and its method of preparation thereof. The formulation acts as a skin disinfectant for all types of wounds. In particular, the antiseptic nano emulsion is an ionic liquid and copper nanocluster-based stable formulation which exhibits both antimicrobial and antiviral activity and is non-toxic.

### FIELD OF THE DISCLOSURE

The disclosure relates to an antiseptic nano emulsion and its methods of preparation thereof. In particular, the disclosure relates to ionic liquids based anti-bacterial, anti-fungal and anti-viral formulation for use as an antiseptic.

### BACKGROUND OF THE DISCLOSURE

Wound antiseptics are indicated for cleaning the skin before a medical procedure such as a blood draw or surgery, for cleaning mucous membranes to treat infections or before using a catheter, for the treatment of critically colonized and infected chronic wounds, to prevent the development of infection in acute wounds with increased risk of infection, such as bites, stabs/punctures, or burns, for decolonization of wounds colonized with MDROs (multi drug resistant organisms), and for the prevention of SSI.

Surgical site infections (SSIs) are infections of the incision, organ or space that occur after surgery. As per the Center for Disease Control and Prevention-USA, surgical site infection (SSI) is associated with a mortality rate of 3%, and 75% of SSI associated deaths are directly attributable to the SSI. Surgical patients initially seen with more complex comorbidities and the emergence of antimicrobial-resistant pathogens increase the cost and challenge of treating SSIs. SSI is the expensive healthcare associated infection type with an estimated annual cost of $3.3 billion and is associated with nearly one million additional inpatient days annually. The use of an antiseptic skin preparation agent before the procedure can reduce the pathogens on the skin surface around the incision.

Therefore, the present disclosure is focused on developing a material that serves as an excellent preoperative antiseptic and helps in preventing post-surgical wound infection.

Staphylococcus aureus, Klebsiella pneumoniae, Escherichia coli and Klebsiella ozaenae are some of the most common infection-causing organisms found in hospitals that have also developed resistance to the antiseptics available in the market. Multidrug-resistant organism (MDRO) infections cause high morbidity and mortality, and excessive costs to patients and hospitals.

Therefore, the second objective of the present disclosure is to develop a material that works against microorganisms that have developed resistance to traditionally available materials.

Some commonly used antiseptics in India are chlorhexidine, chloroxylenol and povidone iodine. Although chlorhexidine is frequently used, it is also known as a common contact allergen. Symptoms include rashes, skin irritation, skin burns, contact dermatitis and even difficulty in breathing. Furthermore, absorption through the skin may potentially lead to systemic toxicity. Even in case of povidone iodine, there have been reports of patients developing systemic iodine toxicity from wounds dressed in gauze soaked in PVP-I or when PVP-I solution was used as a continuous wound irritant.

Hence, the third objective of the present disclosure is focused on developing a newer, safer form of antiseptic material, that can be used safely under diverse situations.

Amongst various biologically active moieties, ionic liquids are compounds with diverse pharmacological activities, including antimicrobial properties. Moreover, water solubility of ionic liquid is an important property that allows us to use water as a base while preparing an ionic liquid based antiseptic entity. Based on these important factors, ionic liquids were selected for the development of antiseptic material. However, to increase the efficacy of the developed ionic liquid, a copper-based nanocluster system has been smartly incorporated within it, so that a synergistic effect is achieved. It is to be noted here that copper nanoparticles are also known to show antimicrobial properties against diverse forms of pathogenic entities. Additionally, the formulation derived from their combination has been stabilized to allow it to be stored for long periods of time without having any effect on the efficacy.

### SUMMARY

It is an object of the present disclosure to provide an antiseptic formulation and a method of preparation thereof.

It is another object of the present disclosure to provide an antiseptic formulation comprises a therapeutically effective amount of: one or more ionic liquids in a concentration of 1.5 to 5.5 wt. %; one or more amines in a concentration of 0.3%-0.75 wt. %; one or more copper salts in a concentration of 0.005%-0.4 wt. %; 1-5 wt. % dimethyl sulphoxide (DMSO); 15-20 wt. % ethanol; 15-20 wt. % glycerol; and 5 wt. % activated charcoal, wherein the antiseptic formulation has antimicrobial and antiviral properties.

According to the present disclosure, 0.005-0.06 M concentration of the antiseptic formulation is suitable for antimicrobial and antiviral properties.

According to the present disclosure, the ionic liquid is selected from the group consisting of Halides of 1-(carboxymethyl)pyridine-1-ium; 1,3-bis(carboxymethyl)-1H-benzo[d] imidazole-3-ium; 3-(carboxymethyl)-1-methyl-1H-imidazol-3-ium and/or the combinations thereof.

According to the present disclosure, the amine is selected from the group consisting of phenylenediamine, ethylenediamine, hydrazine hydrate, diethylenetriamine and/or the combinations thereof.

According to the present disclosure, the copper salt is selected from the group consisting of copper chloride, copper sulfate, copper nitrate, copper acetate and/or their combinations thereof.

According to the present disclosure, there is provided an antiseptic formulation, that is non-toxic and safe to use across a wide range of applications.

It is another object of the present disclosure to provide a method for preparing an antiseptic formulation, comprising the steps of: a) Dissolving 1.5-5.5% concentration of ionic liquid in water mixed alkaline solution of 0.2-0.9% of hydroxybenzaldehyde and 0.3%-0.75% concentration of amine to obtain an ionic solution, wherein the pH of alkaline solution is maintained at 8.0-10.0. b) Heating and continuously stirring the ionic solution obtained in step (a) to produce a clear mixture. c) Adding copper salt at a concentration of 0.005%-0.4% to the clear mixture obtained from step (b) to produce a brown colored Solution A, wherein the said solution comprises 0.1 molar concentration of ionic liquid. d) Mixing 1-5% dimethyl sulphoxide (DMSO), 15-20% ethanol and 15-20% glycerol, followed by homogenization to produce a Solution B. e) Adding 60% of the Solution A, obtained from step (c) with the Solution B, obtained from step (d) to produce Solution C. f) Homogenizing the Solution C for 10 minutes to obtain a dark yellowish-brown solution. g) Adding 5% of charcoal to the Solution C, obtained from step (f) followed by filtration to produce a clear yellowish solution with 0.06 M concentration of antiseptic formulation, wherein the antiseptic formulation has antimicrobial and antiviral activities.

It is yet another object of the present disclosure to provide an antimicrobial and anti-viral formulation for skin disinfection that acts as a disinfectant for all kind of open (traumatic) wounds—abrasions, incisions, lacerations, punctures, avulsions as well as for planned surgical wounds.

It is yet another object of the present disclosure to provide the antibacterial and antifungal agent for surgical preparation to reduce the risk of surgical site infection (SSI).

It is yet another object of the present disclosure to provide a disinfectant solution that has an ionic liquid-based copper nanocluster associated active entity that was found safe for human cells and tends to kill various microbial entities in a very efficient manner.

### BRIEF DESCRIPTION OF DRAWINGS

The foregoing and other features and advantages of the disclosure will be more fully understood from the following descriptions made with reference to the figures.

FIG. 1 illustrates effect of antiseptic formulation on influenza virus H1N1 as (a) Confocal imaging for the cells in case of virus only and antiseptic formulation treated virus and (b) Graphical representation showing the results for the Influenza infection assay with virus (H₁N₁) pre-incubated with different concentrations of antiseptic formulation.

FIG. 2 illustrates Cell viability study using Cell-Titer Blue Cell Viability Assay of final antiseptic formulation on (a) RAW 264.7 cells and (b) HEK cells via graphical representation. After 48 h incubation, the formulation is found to be non-toxic at low concentrations and 70% cell viability is observed at concentration of 0.02 M.

FIG. 3 illustrates particle size of formulation in its control and stabilized forms. The particle size is found to be 803.3 nm (PDI: 0.710) and 441.6 nm (PDI: 0.383) for control formulation (solution A) and stabilized final formulation respectively.

FIG. 4 illustrates zeta potential of control formulation and stabilized final formulation. The zeta potential is found to be +1.41 and −1.43 for control formulation and stabilized formulation respectively.

FIG. 5 illustrates cell viability study using MTT Assay of final formulation on Vero E6 cells via graphical representation. The neat formulation at a concentration of 0.06 M shows a cell viability of 90% demonstrating that the formulation is non-toxic.

FIG. 6 is a bar graph representing cell viability test of formulation 1012-2 (0.02 M final formulation) and 1013-3 (0.0005 M final formulation) in comparison to 10% Betadine (1013-5), 1.5% Savlon (1013-4), 4.8% Dettol (1013-6) and mouthwash (1013-7) on human fibroblasts by LDH (Lactate dehydrogenase assay). Compounds 1013-3 and 1012-2 show minimum cytotoxicity (3.15% and 15.2%, respectively) in comparison to 1013-5 (betadine 27.92% cytotoxicity). Other compounds (1013-4, 1013-6 and 1013-7) show very high cytotoxicity with LDH assay.

FIG. 7 illustrates field-emission scanning electron micrographs of E. coli ATCC 25922 after treatment with final formulation at 0.5×MIC (B) and 1×MIC (C) compared with the untreated controls (A). The images are taken at different magnification levels as mentioned in the images. Compared to the untreated control, extensive cell damage, cell debris and loss of cell architecture are observed upon treatment with formulation at MIC concentration (0.02 M). Upon treatment with 0.5x MIC concentration (0.01 M), surface blebbing and/or shriveling was noticed. Additionally, the mean cell length is significantly smaller (p<0.001) in the sample treated with 0.5×MIC (0.01 M) formulation (1.20+/−0.40 μm) than the untreated controls (2.18+/−0.52 μm).

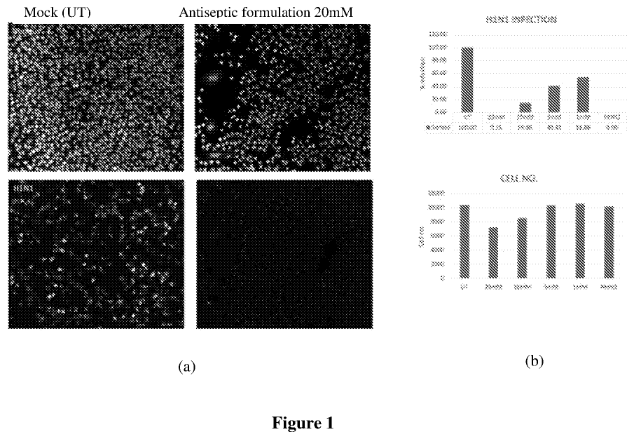

### DETAILED DESCRIPTION OF DISCLOSURE

Reference will be made in detail to embodiments of the present disclosure. The same or similar elements and the elements having same or similar functions are denoted by reference numerals throughout the descriptions. The embodiments described herein with reference to drawings are explanatory, illustrative, and used to generally understand the present disclosure. The embodiments shall not be construed to limit the present disclosure.

The present disclosure provides for antiseptic formulation having antimicrobial and antiviral properties.

According to embodiments of the present disclosure, it provides for ionic liquid and copper nanocluster based antimicrobial and anti-viral formulations for skin disinfection.

According to another embodiment of present disclosure, the antiseptic formulation comprises ionic liquids, amines and copper salts.

The ionic liquids according to the embodiment of the present disclosure can be selected from the group consisting of Halides of 1-(carboxymethyl)pyridine-1-ium; 1,3-bis (carboxymethyl)-1H-benzo[d]imidazole-3-ium; 3-(carboxymethyl)-1-methyl-1H-imidazol-3-ium and/or their combinations thereof.

The amines according to the embodiment of the present disclosure are, but not limited to, phenylenediamine, ethylenediamine, hydrazine hydrate, diethylenetriamine and/or their combinations thereof.

The copper salts according to the embodiment of the present disclosure are, but not limited to, copper chloride, copper sulfate, copper nitrate, copper acetate and/or their combinations thereof.

According to another embodiment, the present disclosure provides a method of preparation of the antiseptic formulation. The method involves the ionic liquids that are added in a concentration of 1.5-5.5% and are dissolved in water mixed with alkaline solution (pH 8.0-10.0) of hydroxybenzaldehyde (0.2-0.9%) and the concentration of 0.3%-0.75% of amines. The obtained solution is heated and continuously stirred until it becomes sufficiently clear, and further copper salts are added at a total concentration below 0.4% to obtain a brown colored solution. The solution is made in such a way that it produces 0.1 molar concentration of ionic liquid. This is solution A. To prepare solution B, 1-5% dimethyl sulphoxide (DMSO) and 15-20% ethanol is kept in a beaker and mixed on a magnetic stirrer without heating. This mixture is homogenized under a high-speed homogenizer for 2 minutes. Then, 15-20% glycerol is added dropwise followed by homogenization again. This is solution B. 60% of solution A is added to solution B to produce solution C. Solution C is homogenized for 10 minutes. A dark yellowish-brown colored solution is obtained. Finally, 5% charcoal is added to solution C and this formulation is filtered via Buchner funnel vacuum filtration. A clear yellowish solution is obtained having concentration of 0.06 M which is the final stabilized antiseptic formulation.

The excipients used for stabilization as well as for ensuring proper absorption of the formulation are described below.

Dimethylsulfoxide (DMSO), a colorless organosulfur solvent, enhances percutaneous penetration when used in combination with other substances. DMSO is also used topically for wound recovery, and it also has been studied to have some antiviral effects. To produce this novel formulation, DMSO is chosen to facilitate the transdermal delivery.

Ethanol, a topical penetration enhancer can enter the skin and removes measurable quantities of the lipid barrier material from the stratum corneum. This lipid extraction may lower the skin barrier function and render the membrane more permeable. Hence, ethanol facilitates the solute in the formulation.

Glycerin is used in topical solutions as a humectant, which can attract, bind, and hold moisture to the site of application, and thus it provides protection against skin irritants, accelerates the wound-healing processes. Glycerin is also considered as a mucosal adherent. The effective concentration of glycerin is required to produce the novel formulation.

Activated charcoal has a porous texture and it is used to remove the undesirable odor and color of the formulation for the purpose of stabilizing it. It is important to use the right concentration of charcoal to produce the desired formulation. Higher concentration reduces the therapeutic value of the active ingredient while with the lesser concentration, either the stability of the formulation is compromised, or the undesirable characteristics persist.

The prepared antiseptic formulation has been fully characterized and confirmed by Particle Size and Polydispersity index (PDI), Zeta Potential, Nuclear Magnetic Resonance (NMR), High Resolution transmission electron microscopy (HR-TEM) and Field Emission scanning electron microscopy (FE-SEM) techniques.

The generation of copper nanoclusters is established by using spectroscopic methods, size was determined using dynamic light scattering based particle size analyzer and formulation was optimized using zeta potential.

The present disclosure is focused on developing a newer, safer and stable form of antiseptic formulation that can be used safely under diverse situations. The formulation of the present disclosure besides being anti-virulent and antimicrobial in its properties, is non-toxic in nature when compared to other known antiseptics.

### Physical Properties of the Formulation:

The mean diameters of the control formulation (Solution A, as mentioned before) and final stabilized forms are measured with appropriate dilutions with distilled water (1:20) using Delsa™ Nano C (Beckman Coulter, California, USA). Zeta potential is also determined by using Delsa™ Nano C (Beckman Coulter, California, USA).

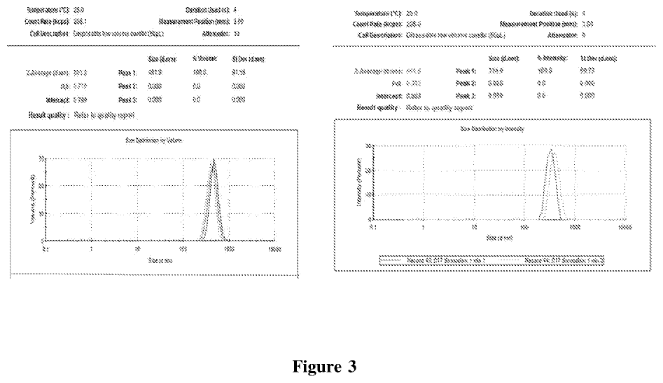

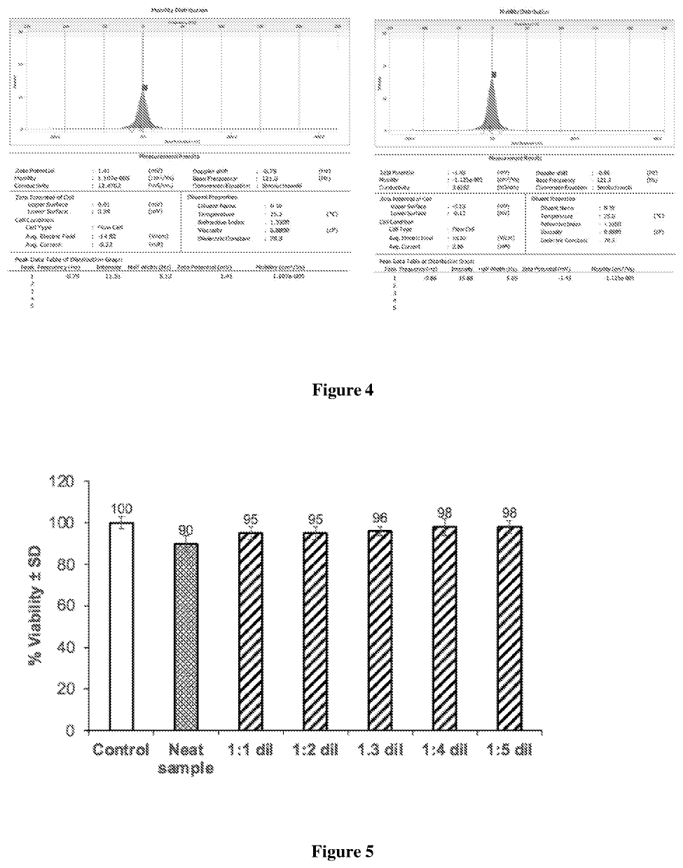

The particle size is found to be 803.3 nm (PDI: 0.710) and 441.6 nm (PDI: 0.383) for Control formulation and final stabilized formulation respectively (FIG. 3). The zeta potential is found to be +1.41 and −1.43 for Control formulation and final formulations respectively (FIG. 4).

Control formulation and final stabilized formulation are observed microscopically using HRTEM (JEOL-JEM 2100 Plus) for uniformity of size, shape and physical stability characteristics i.e., aggregation or irregularity, at an accelerated voltage of 80 to 200 kV. A drop of the sample appropriately diluted is placed on a carbon-coated copper grid to leave a thin film on the grid. Excess of the solution is drained off with a filter paper. The grid is air-dried thoroughly, and samples are viewed under HRTEM for various morphological attributes and particle size.

The morphology of samples is spherical. The particle size, observed via HRTEM, is 200 nm for final formulation and is around 500 nm for control formulation.

Field emission scanning electron microscope (FESEM) is used for assessing the morphological characteristics of control formulation and final formulations. The surface texture and morphology of the samples are studied using FESEM, (HITACHI SU6600). Before observation, the samples are fixed on a double adhesive carbon tape, which is stuck on aluminum stubs and then coated with gold under an argon atmosphere. The samples are visualized by FESEM with an accelerating voltage of 0.5-30 kV. FESEM characterization shows the network structure of final formulation and clear morphology due to its higher resolution. The formulation is found to be distributed evenly throughout the matrix.

### Cell Viability Studies:

The following study is done to evaluate the toxicity of the antiseptic formulation according to the embodiments of the present disclosure.

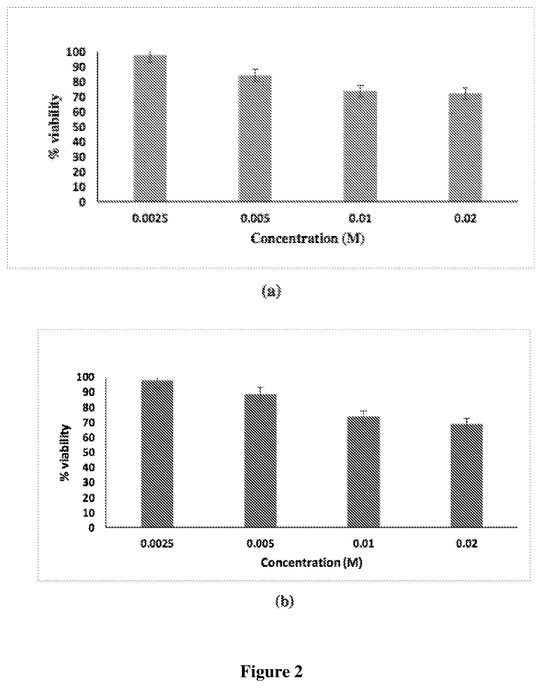

The RAW 264.7 and HEK cells are seeded and cultured for 24 h control to treatment with formulation at concentrations ranging from 0.0025 to 0.02 M. Cells containing media without a drug are used as a control. Cell-Titer Blue® Cell Viability Assay is used to determine the viability after 48 h incubation with treatment. Absorbance response for this assay is quantified at a Amax of 570 nm after 1 h incubation (FIGS. 2a and 2b). The final formulation is found to be non-toxic at low concentration while 70% cell viability was observed at a concentration of 0.02 M for both cell lines which demonstrates very low toxicity.

The MTT (3-(4,5-dimethylthiazol-2-yl)-2,5-diphenyltetrazolium bromide) assay is used to test in-vitro cytotoxicity of formulation. VeroE6 cells are grown in Dulbecco's modified Eagle's medium supplemented with fetal calf serum and antibiotic-antimycotic in a humidified incubator with 5% CO₂ supply at 37° C. Cells are grown up to 80-90% confluency and harvested with trypsin-EDTA and plated in a 96-well plate at 1×104 cells/well and allowed to grow for 24 h before treatment. After 24 h, the media is replaced with neat samples and the samples are diluted 1-5 times in media. Post-treatment, media and media containing samples are replaced with MTT reagent and allowed to incubate for 4 h. Formazan crystals are solubilized by adding DMSO into each well and the absorbance is taken at 570 nm in a microplate reader. The cytotoxicity is expressed as percentage cell viability. Each experiment is performed in triplicates (n=3). The neat formulation at a concentration of 0.06 M shows a cell viability of 90%, demonstrating that the formulation is non-toxic (FIG. 5).

The cell viability test of formulation 1012-2 (0.02 M formulation) and 1013-3 (0.0005 M formulation) in comparison with 10% Betadine (1013-5), 1.5% Savlon (1013-4), 4.8% Dettol (1013-6) and Cetylpyridinium based mouthwash (1013-7) on human fibroblasts by LDH (Lactate dehydrogenase assay).

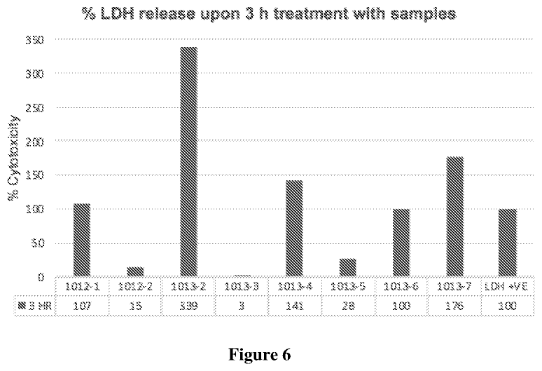

Fifty thousand normal human dermal fibroblast (NHDF) cells are seeded per well in a trans-well plate. Compounds are diluted in D-MEM (Invitrogen) as recommended. 500 μL of respective diluted compounds are added to the bottom well in the trans-well plate and 200 μL of DMEM is added to the top well followed by incubation in CO₂ incubator for 3 hours. Lysis buffer from the assay kit is added to one of the bottom well 30 minutes before the assay. Media from top well is collected and added to 96 well flat bottom plate (50 μL per well in triplicate). 50 μL of substrate is added to each well and is incubated at 25° C. for 20 minutes. 50 μL of stop solution is added to each well. Absorbance is read at 490 nm. Percentage cytotoxicity is calculated by normalizing values with lysis buffer (LDH+ve 100% cytotoxicity). Compounds 1013-3 and 1012-2 show minimum cytotoxicity (3.15% and 15.2%, respectively) in comparison with 1013-5 (betadine 27.92% cytotoxicity). Other compounds (1013-4, 1013-6 and 1013-7) show very high cytotoxicity with LDH assay (FIG. 6).

The antiseptic formulation or solution of the present disclosure is focused on developing a material that prevents the spread of bacterial and fungal infections. The efficacy of the disclosure was determined by means of the following examples:

### Efficacy Study:

The antiseptic formulation according to the embodiments of the present disclosure is further used to assess its virucidal activity on influenza A viruses (IAVs). To check the virucidal activity of antiseptic solution, the IAV infection assay is performed with viruses treated with 1 mM, 5 mM, 10 mM and 20 mM. The IAV infection assay involves infecting human lung epithelial cell line A549 for 10 hours, followed by immunostaining of the viral nucleoprotein (NP) for microscopy. To count the number of cells, DAPI is used which stains the cell nuclei. Ammonium chloride (NH₄Cl) is used as positive control in the experiments as NH₄Cl is a tested inhibitor of IAV infection. The infected cells are detected using high-content confocal microscopy, and the percentage of infected cells is calculated by the fraction of NP-infected cells. Incubating the antiseptic formulation sample at a concentration of 20 mM inhibited IAV infection by >99%. Incubating IAV with different concentrations of antiseptic solution indicated, 20 mM as the optimal concentration at which IAV infection is almost blocked.

The antiseptic solution/formulation according to the other embodiments of the present disclosure is further used to assess its fungicidal activity on Candida albicans ATCC 90028. To check for Antifungal Susceptibility Testing, Broth Microdilution Assay is performed. Dulbecco's Modified Eagle's medium (DMEM) with glutamine, and 4.5 g/l glucose is used as the culture medium. The final C. albicans inoculum density is 0.5 to 2.5×103 cells/ml (e.g., milliliter). The test substance is prepared as a two-fold dilution series and tested at a final concentration range of 0.02 M to 0.0003 M. The microtiter plates are incubated at 37° C. for 24h. Amphotericin B is taken as the positive control. The MIC or minimum inhibitory concentration (concentration at which complete inhibition of microbial growth is observed) is found to be 0.01 M in case of antiseptic formulation.

Antifungal Susceptibility Testing is also performed on other fungal varieties like Rhizopus arrhizus CI-4 and S-1 and Aspergillus fumigatus CI-1 and CI-2 and the MIC of the formulation against both species is found to be 0.005 M.

The antiseptic formulation according to yet another embodiment of the present disclosure is further used to assess its bactericidal activity on Escherichia coli ATCC 25922. To check for Antibacterial Susceptibility Testing, Broth Microdilution Assay is performed. Dulbecco's Modified Eagle's medium (DMEM) with glutamine, and 4.5 g/l glucose is used as the culture medium. The test inoculum is prepared by colony suspension method, with the final inoculum density being 5×10⁵ cfu/ml. The test substance is prepared as a two-fold dilution series and tested at a final concentration range of 0.02 M to 0.00125 M. The microtiter plates are incubated at 37° C. for 16-20 h. 1 μg/ml Amikacin is taken as the positive control. The MIC or minimum inhibitory concentration (concentration at which complete inhibition of microbial growth is observed) is found to be 0.02 M in case of antiseptic formulation.

Using Broth Microdilution Assay, the formulation's efficacy is tested against other gram negative and gram-positive bacterial species. Stenotrophomonas maltophilia is a globally emerging gram-negative multi drug resistant organism. The MIC of the formulation is found to be 0.02 M in case of S. maltophilia MTCC 434 (ATCC 13637). Acinetobacter baumannii is a gram-negative opportunistic pathogen that is a common cause for hospital derived infections. Complete inhibition of A. baumannii MTCC 12889 by the formulation is observed at 0.01 M. Methicillin resistant Staphylococcus aureus (MRSA) is a group of gram-positive bacteria that cause many difficult to treat infections in humans. The MIC of the formulation is 0.005 M against S. aureus CI-4 MRSA.

The antibiofilm activity of the antiseptic against S. aureus ATCC 29213 and C. albicans ATCC 90028 biofilms is determined in 96 well flat-bottomed microtiter plates by XTT dye reduction test. For prevention studies, 200 μL (e.g., micro liter) of the microbial suspension (S. aureus˜107 cfu/ml; C. albicans˜106 cells/ml) is incubated for biofilm formation in the nutrient medium [S. aureus, Mueller Hinton Broth (MHB); C. albicans, RPMI-1640] containing two-fold serial dilutions of the test substance (0.06 M and 0.04 M to 0.0003 M) for 24 h at 37° C. Untreated control is set up in parallel. After incubation, the formed biofilms are washed twice with normal saline and exposed to XTT-menadione solution (XTT, 0.5 mg/ml; menadione, 10 UM (e.g., micromole)) for 1 h at 37° C. in the dark. The absorbance of the supernatants is measured at 450 nm. Likewise, to determine the efficacy of the test substance in eradicating pre-existing biofilms, 24 h old biofilms of S. aureus ATCC 29213 and C. albicans ATCC 90028 are treated with two-fold serial dilutions of the test substance for 24 h at 37° C., and quantified by XTT dye reduction test. MBPC50, MBPC90 and MBPC100 are defined as concentrations of the test substance that led to 50%, 90% and 100% decrease in biofilm formation respectively relative to the untreated controls. MBIC50, MBIC90 and MBIC100 are defined as the concentrations of test substance that lead to 50%, 90% and 100% reduction in pre-formed biofilm biomass respectively compared with the untreated controls. At 0.03 M, the formulation prevents the formation of S. aureus biofilm by 84% and C. albicans biofilm by 67%. At 0.06 M, the formulation prevents the formation of S. aureus biofilm by 92% and C. albicans biofilm by 85%. At 0.06 M, the formulation inhibits pre-formed S. aureus biofilm by 92% and C. albicans biofilm by 64%.

### Risk of Microbial Resistance:

The risk of development of microbial resistance to the formulation is tested in S. aureus CI-4 (MRSA) by modifying the method of Tambe et al. (2001). Dulbecco's Modified Eagle's medium (DMEM) with glutamine, and 4.5 g/l glucose and 5% Luria broth is used as the culture medium. Broth microdilution assay is performed using the formulation at concentrations corresponding to three doubling dilutions above to three doubling dilutions below the MIC. The final S. aureus inoculum density in the experimental set-up is 5×10⁵ cfu/ml. After incubation at 37° C. for 16-20 h, the culture below the MIC tube (sub-MIC) is used to prepare the inoculum for the next transfer. The experiment is conducted up to 20 passages. Untreated control is also passaged in parallel, and its MIC determines after 10th and 20th passage. The MIC or minimum inhibitory concentration (concentration at which complete inhibition of microbial growth is observed) is 0.005 M pre-passage, 0.005 M post 10th passage and 0.005 M post 20th passage. Hence. No change in MIC of the formulation against S. aureus is noted after exposure to the compound at sub-MIC up to 20 passages demonstrating no microbial resistance to the formulation.

Using the same assay, the risk of microbial resistance in betadine has been compared with the antiseptic formulation. The MIC of betadine against S. aureus CI-4 (MRSA) is 0.125% w/v pre-passage which increased to 0.25% w/v at the 5th passage which further increased to 0.5% w/v at the 18th passage. Therefore, some bacterial resistance in the case of betadine has been observed.

### Stability Study:

To check for stability, 2 samples of the formulation are preserved at room temperature and at 4° C. respectively. Both samples are observed monthly to check for change in physical factors like color, odor, etc. and for efficacy against bacterial and fungal species of E. coli and C. albicans using Broth Microdilution Assay specified in (B) and (C) above. The antimicrobial activity along with the physical characteristics of the formulation is checked at 1, 2, 3, 4, 5 and 6 months. The MIC of the formulation against E. coli is remained constant at 0.02 M while the MIC of formulation against C. albicans has been increased from 0.005 M in month 1 to 0.02 M in month 6. There is no change in color, pH or odor and no precipitation of the contents has been observed.

### Time-Kill Assay:

The killing kinetics of 0.06 M formulation using Time-Kill Procedure according to ASTM 2315-16 are determined against bacterial strain E. coli (ATCC 25922) at different time intervals (30 seconds, 1 minute, 2 minutes, 5 minutes and 10 minutes). The test inoculum is prepared by the colony suspension method and added to formulation of 0.06 M, such that the volume of inoculum suspension is 5% of the total test volume and the final inoculum density in the experimental set-up is 1×10⁶ cfu/ml. The test set-ups are incubated at 37° C. Post-addition of culture to the products, contact time intervals of 30 seconds, 1 minute, 2 minutes, 5 minutes and 10 minutes are maintained. After each time interval, an aliquot of the test sample is collected, added to Dey Engley neutralizing broth, followed by preparation of ten-fold serial dilutions in normal saline. The viable organisms are recovered and quantified by drop plating on Soyabean-casein digest agar in triplicate after 24 h of incubation at 37° C. After incubation, the plates are observed for recovery of the test organism. Time zero and untreated controls (in diluent blank) are set up in parallel.

The Log 10 cfu/ml value at time 0 is 6.14+/−0.06 which is reduced to 5.22+/−0.02 at 30 seconds and 3.85+/−0.2 at 1 minute. Complete killing is observed at 2 minutes.

### Mechanism of Action:

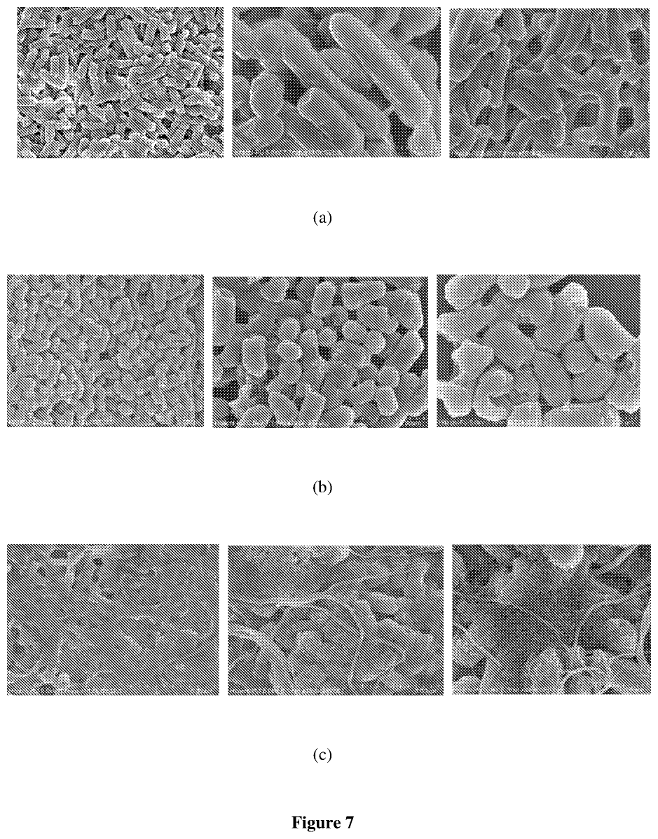

The impact of the formulation on bacterial cell surface is evaluated using FE-SEM. Dulbecco's Modified Eagle's medium (DMEM) with glutamine, and 4.5 g/l glucose is used as the culture medium. The test inoculum is prepared by the colony suspension method, with the final inoculum density in the experimental setup being 5×10⁵ cfu/ml. The bacterial cell suspension is treated with the test substance at 0.5× and 1×MIC for 18 h at 37° C. The untreated controls are set up in parallel. The resulting suspensions are centrifuged at 10,000 rpm for 10 minutes, the pellets are washed twice with phosphate buffered saline and the samples are processed for Field-emission Scanning Electron Microscopy (FE-SEM). Briefly, the samples are fixed with 2.5% glutaraldehyde at 4° C., washed thrice in the rinsing buffer [Sorensen's phosphate buffer (0.1 M, pH 7.2) with 7% sucrose], and dehydrated in a series of ethanol solutions (50%, 10 min; 70%, 10 min; 80%, 15 min; 90%, 15 min; and 100%, 20 min). The resulting specimens are coated with platinum, and observed under FE-SEM (SU 8010, Hitachi, Japan). Typical rod-shaped cells are seen in the untreated E. coli, with an average length of 2.18+/−0.52 um and breadth of 0.61+/−0.07 um. Compared with the untreated control, extensive cell damage, cell debris and loss of cell architecture is observed upon treatment with formulation at MIC concentration (0.02 M). Upon treatment with 0.5×MIC concentration (0.01 M), surface blebbing and/or shriveling is noticed. Additionally, the mean cell length is significantly smaller (p<0.001) in the sample treated with 0.5x MIC (0.01 M) formulation (1.20+/−0.40 um) than the untreated controls (2.18+/−0.52 um). The cell damage and debris are comparatively lesser in E. coli treated with formulation at 0.5×MIC concentration than at 1×MIC (FIG. 7).

1. An antiseptic formulation comprising a therapeutically effective amount of:
one or more ionic liquids in a concentration of 1.5 to 5.5 wt. %; one or more amines in a concentration of 0.3%-0.75 wt. %; one or more copper salts in a concentration of 0.005%-0.4 wt. %; 1-5 wt. % dimethyl sulphoxide (DMSO); 15-20 wt. % ethanol; 15-20 wt. % glycerol; and 5 wt. % activated charcoal,
wherein the antiseptic formulation has antimicrobial and antiviral properties.

2. The formulation as recited in claim 1, wherein 0.005-0.06 M concentration of the antiseptic formulation is suitable for antimicrobial and antiviral properties.

3. The formulation as recited in claim 1, wherein the ionic liquid is selected from the group consisting of Halides of 1-(carboxymethyl)pyridine-1-ium; 1,3-bis(carboxymethyl)-1H-benzo[d]imidazole-3-ium; 3-(carboxymethyl)-1-methyl-1H-imidazol-3-ium and/or the combinations thereof.

4. The formulation as recited in claim 1, wherein the amine is selected from the group consisting of phenylenediamine, ethylenediamine, hydrazine hydrate, diethylenetriamine and/or the combinations thereof.

5. The formulation as recited in claim 1, wherein the copper salt is selected from the group consisting of copper chloride, copper sulfate, copper nitrate, copper acetate and/or their combinations thereof.

6. The formulation as recited in claim 1 is non-toxic.

7. A method for preparing an antiseptic formulation, comprising the steps of:
a) Dissolving 1.5-5.5% concentration of ionic liquid in water mixed alkaline solution of 0.2-0.9% of hydroxybenzaldehyde and 0.3%-0.75% concentration of amine to obtain an ionic solution, wherein the pH of alkaline solution is maintained at 8.0-10.0.
b) Heating and continuously stirring the ionic solution obtained in step (a) to produce a clear mixture;
c) Adding copper salt at a concentration of 0.005%-0.4% to the clear mixture obtained from step (b) to produce a brown colored Solution A, wherein the said solution comprises 0.1 molar concentration of ionic liquid;
d) Mixing 1-5% dimethyl sulphoxide (DMSO), 15-20% ethanol and 15-20% glycerol, followed by homogenization to produce a Solution B;
e) Adding 60% of the Solution A, obtained from step (c) with the Solution B, obtained from step (d) to produce a Solution C;
f) Homogenizing the Solution C for 10 minutes to obtain a dark yellowish-brown solution;
g) Adding 5% of charcoal to the Solution C, obtained from step (f) followed by filtration to produce a clear yellowish solution of antiseptic formulation,
wherein the antiseptic formulation has antimicrobial and antiviral activities.

8. The method, as recited in claim 7, wherein the ionic liquid is selected from the group consisting of Halides of 1-(carboxymethyl)pyridine-1-ium; 1,3-bis (carboxymethyl)-1H-benzo[d]imidazole-3-ium; 3-(carboxymethyl)-1-methyl-1H-imidazol-3-ium and/or the combinations thereof.

9. The method, as recited in claim 7, wherein the amine is selected from the group consisting of phenylenediamine, ethylenediamine, hydrazine hydrate, diethylenetriamine and/or the combinations thereof.

10. The method, as recited in claim 7, wherein the copper salt is selected from the group consisting of copper chloride, copper sulfate, copper nitrate, copper acetate and/or their combinations thereof.

11. The method as recited in claim 7, wherein 0.005-0.06 M concentration of the antiseptic formulation is non-toxic and suitable for antimicrobial and antiviral properties.
